(19) United States
(12) Patent Application Publication
(10) Pub. No.: US 2006/0243027 A1
(43) Pub. Date: Nov. 2, 2006

(54) Package for a sensing element, retaining device for retaining the sensing element in the package, sensor, and methods of making the same

(76) Inventors: Charles Scott Nelson, Fenton, MI (US); Paul Hugo Ruterbusch, Flushing, MI (US); Daryl Harris, Leudelange (LU); Remi B. Loevenbruck, Habay La Neuve (BE); Frank J. Ruzicka, Flint, MI (US); Dana M. Serrels, Davison, MI (US); Dennis E. Nowlen, Burton, MI (US); Richard F. Beckmeyer, Davisburg, MI (US)

Correspondence Address: Jimmy L. Funke;Delphi Technologies, Inc., M/C 480-410-202, P.O. Box 5052, Troy, MI, 48007, US

(21) Appl. No.: 11/117,889

(22) Filed: Apr. 29, 2005

(51) Int. Cl. G01N 33/497

(52) U.S. Cl. 73/23.31

(57) ABSTRACT

A packaged sensor can comprise: a housing, a retaining device disposed in the housing, a sensing element, a sealing member disposed in between the retaining device and the housing terminal end, a first electrical lead, a second electrical lead, and a first support member. The first support member can be disposed between the sealing member and the housing terminal end. The retaining device can be disposed in a chamber in the housing, and can comprise retaining device comprising a first end and a second end, channels extending between the first end and the second end, and can have a sealant material disposed at the first end of the retaining device. The sensing element can have an element terminal end disposed in physical contact with the retaining device. The first electrical lead and a second electrical lead can be disposed in electrical communication with the element terminal end, can extend from the element terminal end, through the channels in the retaining device, and can extend from the second end of the retaining device.

### TECHNICAL FIELD

The present disclosure is related to a package for a sensing element, a retaining device for retaining the sensing element in the package, a sensor, and methods of making the same and, more particularly, to a package, retaining device and sensor adapted for use in a vehicle exhaust system, and methods of making the same.

### BACKGROUND

As environmental concerns and the demand for improved automobile fuel efficiency have increased, more stringent emission regulations have been implemented. The automobile industry has responded by developing improved exhaust treatment components. As a result, many vehicle exhaust systems typically comprise one or a variety of components designed to reduce undesirable emissions and/or to improve fuel efficiency, or to assist in the foregoing. Examples of such components comprise, but are not limited to, catalytic converters, catalytic absorbers, diesel particulate traps, non-thermal plasma conversion devices, and the like.

Vehicle exhaust environments can reach temperatures in excess of about 1,000° C. Therefore, it is desirable that components used in an exhaust system environment, such as sensors, should be capable of withstanding such temperatures without physical destruction or performance degradation.

### SUMMARY

Disclosed herein, in one embodiment, is a packaged sensor comprising a housing defining a housing sensing end, a housing terminal end opposite the housing sensing end, and a chamber between the housing sensing end and the housing terminal end. The packaged sensor also comprises a retaining device disposed in the chamber. The retaining device comprises a first end and a second end, a first channel extending between the first end and the second end, and a sealant material disposed at the first end of the retaining device. The packaged sensor also includes a sensing element comprising a sensing element terminal end disposed in physical contact with the retaining device, a sealing member disposed in the chamber between the retaining device and the housing terminal end, and a first support member disposed between the sealing member and the housing terminal end. A first electrical lead and a second electrical lead are disposed in electrical communication with the sensing element terminal end, and extend from the element terminal end, through the retaining device, and from the second end of the retaining device.

In another embodiment, a method of making a packaged sensor is disclosed. The method also comprises disposing a retaining device in a chamber of a housing, wherein the housing defines a housing sensing end, a housing terminal end opposite the housing sensing end, and a chamber between the housing sensing end and the housing terminal end, and wherein the retaining device comprises a first end and a second end, a first channel extending between the first end and the second end, and a sealant material disposed at the first end of the retaining device. The method also comprises disposing a sensing element comprising a sensing element terminal end in physical contact with the retaining device, disposing a sealing member in the chamber between the retaining device and the housing terminal end, disposing a first support member between the sealing member and the housing terminal end, and disposing a first electrical lead and a second electrical lead in electrical communication with the sensing element terminal end, such that the first and second electrical leads extend from the element terminal end, through the retaining device, and from the second end of the retaining device. The method also comprises forming a sealing member from the sealing member precursor by exerting a longitudinal force on the sealing member precursor.

The above described and other features are exemplified by the following figures and detailed description.

### DRAWINGS

Refer now to the figures, which are exemplary embodiments, and wherein like elements are numbered alike.

FIG. 1 is a side view of an exemplary sensor.

FIG. 2 is a cross-sectional side view of the sensor of FIG. 1.

FIG. 3 is a side view of a retaining device contained in the sensor shown in FIG. 1.

FIG. 4 is a cut-away perspective view of a terminal end of the sensor in FIG. 1.

FIG. 5 is a cut-away perspective view of a sensing end of the sensor shown in FIG. 1.

### DETAILED DESCRIPTION

At the outset of the detailed description, it should be noted that the terms “first,” “second,” and the like herein do not denote any order or importance, but rather are used to distinguish one element from another, and the terms “a” and “an” herein do not denote a limitation of quantity, but rather denote the presence of at least one of the referenced items. Similarly, it is noted that the terms “bottom” and “top” are used herein, unless otherwise noted, merely for convenience of description, and are not limited to any one position or spatial orientation. In addition, the modifier “about” used in connection with a quantity is inclusive of the stated value and has the meaning dictated by the context (e.g., includes the degree of error associated with measurement of the particular quantity). Unless defined otherwise herein, all percentages herein mean weight percent (“wt.%”). Furthermore, all ranges disclosed herein are inclusive and combinable (e.g., ranges of “up to about 25 weight percent (wt.%), with about 5 wt.% to about 20 wt.% desired, and about 10 wt.% to about 15 wt.% more desired,” are inclusive of the endpoints and all intermediate values of the ranges, e.g., “about 5 wt.% to about 25 wt.%, about 5 wt.% to about 15 wt.%”, etc.). Finally, unless defined otherwise, technical and scientific terms used herein have the same meaning as is commonly understood by one of skill in the art to which this invention belongs.

Disclosed herein is a package for a sensing element, a retaining device for retaining the sensing element in the package, a sensor, and methods of making the same. The present disclosure is applicable to various sensing elements including, but not limited to, temperature sensors and gas sensors such as oxygen sensors, ammonia sensors, nitrogen oxide sensors, hydrogen sensors, hydrocarbon sensors, and the like. In addition, although the packaged sensor is described herein in connection with a planar sensing element, it is to be understood that other types of sensing elements can be disposed in the package with minor adjustments such as, for example, wide-range, switch-type, and the like.

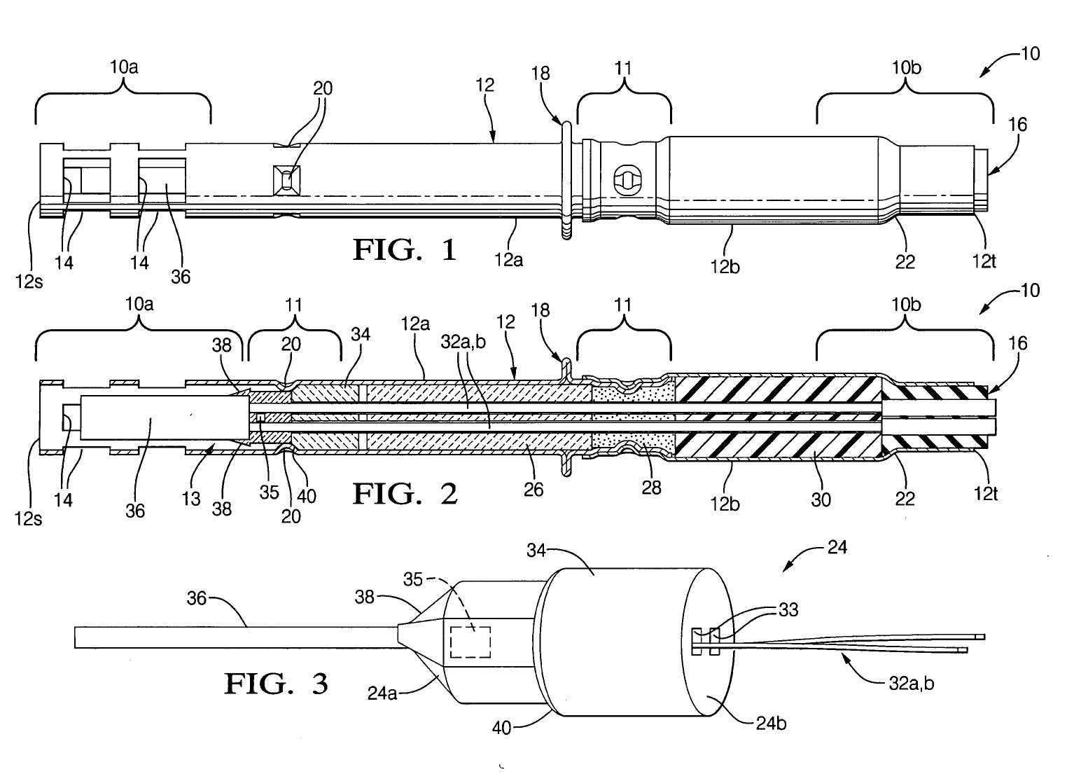

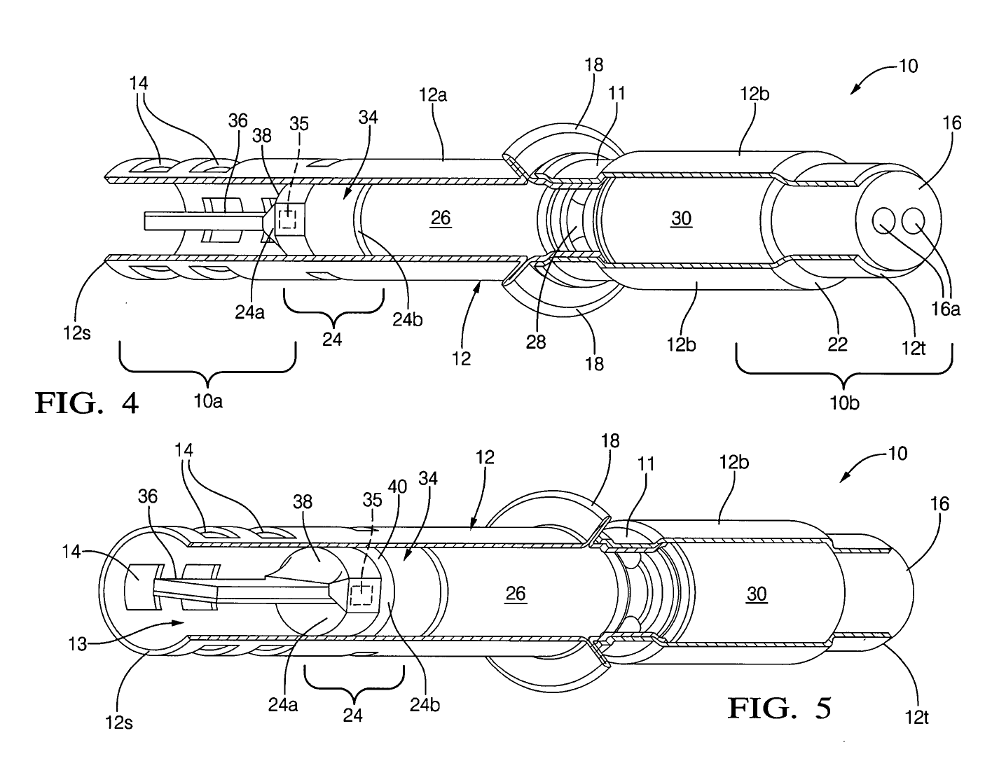

FIG. 1 shows a packaged sensor 10, which can comprise a sensing end 10a and a terminal end 10b opposite the sensing end 10a, and which can comprise a housing 12 with a generally longitudinal shape defining a first end 12s and a second end 12t. It should be understood that the housing 12 can comprise a single, unitary member or, alternatively, the housing 12 can comprise two or more joined sections. For example, as shown in FIG. 1, housing 12 can comprise a lower housing section 12a and an upper housing section 12b. The lower housing section 12a can be sized and dimensioned to fit concentrically within the upper housing section 12b or, alternatively, the upper housing 12b can be sized and dimensioned to fit concentrically within the lower housing section 12a. Alternatively, a joining section (not shown) could be disposed between sections 12a,b. Although illustrated herein as cylindrical, it should be understood that the housing 12 and/or housing sections 12a,b can comprise any shape or size depending upon various factors such as, but not limited to, the length of the components contained within the housing (e.g. the sensing element, and the like), the degree of insertion into, for example, an exhaust pipe, and/or the like. For example, by increasing the length of the housing 12, a greater portion of the sensor 10 can be inserted into, for example, an exhaust pipe, which can provide a greater degree of flexibility in sampling the exhaust gas.

The lower housing section 12a can comprise a plurality of openings 14 at the lower housing section 12a, for providing fluid communication between the interior of the housing and the environment in which it can be disposed when in use. Thus, when the packaged sensor is in use, a fluid to be sensed (for example, a gas) can flow into the housing 12 at the sensing end 10a through the openings 14.

Lower housing section 12a can comprise a lower retention member 20 which can be disposed between openings 14 and the upper housing section 12b. The lower housing section 12a and the lower retention member 20 can comprise a single, unitary member, or it can comprise two or more pieces that are joined together (e.g., lower retention member 20 can be joined to the lower housing section 12a rather than being continuous with the lower housing section 12a). As shown, the lower retention member 20 comprises a plurality of radially spaced apart crimped sections, but it should be understood that it can comprise any structure capable of limiting the movement of components disposed in the packaged sensor 10.

The upper housing section 12b can comprise an opening 15 at the terminal end 10b in which a seal 16 can be disposed for retaining and/or supporting the various components disposed in the packaged sensor 10. The seal 16 can comprise two or more spaced apart openings 16a,b, in which electrical leads 32a,b can be disposed, supported and maintained in spaced relation to one another.

The upper housing section 12b can comprise an upper retention member 22 disposed adjacent to the seal 16. The upper retention member 22 and the upper housing section 12b can comprise a single, unitary member, or the upper retention member 22 and the upper housing section 12b can comprise two or more pieces that are joined together (e.g., upper retention member 22 can be joined to the upper housing section 12b rather than being continuous with the upper housing section 12b). As shown, the upper retention member 22 comprises a shoulder tapered toward the seal 16, but it should be understood that it could comprise any structure that can be capable of limiting the movement of components that can be disposed in the packaged sensor 10.

Housing 12 can comprise a region 11 disposed between the sensing end 10a and the terminal end lob. Region 11 by, for example, crimping the housing 12 can be formed after disposition of the sensor components into the housing 12 by, for example, crimping the housing 12.

A flange 18 can be disposed between the sensing end 10a and the terminal end 10b of the packaged sensor 10, such that it extends radially from the exterior surface of the housing 12. The flange 18 and the housing 12 can comprise a single, unitary member, or they can comprise two or more pieces that are joined together (e.g., the flange 18 can be joined to the housing 12 rather than being continuous with the housing 12). Although illustrated herein as being disposed on the lower housing section 12a, it should be understood that the flange 18 could be disposed on either the lower housing section 12a or the upper housing section 12b. Flange 18 can be adapted to be disposed between an exhaust mount (not shown) and a threaded screw or nut (not shown), for mounting to, for example, a vehicle exhaust system.

Although the region 11 is shown herein as disposed on upper housing section 12b, and radially extending flange 18 is shown herein as disposed on lower housing section 12a, it should be understood that either component can be disposed on the lower housing section 12a or the upper housing section 12b. Moreover, it also should be understood that the relative positions of the crimped region 11 and the radially extending flange 18 may be changed, for example, the radially extending flange 18 can be disposed upstream of the crimped region 11.

FIG. 2 shows various exemplary components disposed in the housing 12. As shown, a retaining device 24 can be disposed adjacent to the openings 14 at the sensing end 10a; a first support member 30 can be disposed at the terminal end 10b, adjacent to the seal 16; and a sealing member 28 (e.g. talc, glass, and/or the like) can be disposed adjacent to the retaining device 24 and the crimped region 11, between the retaining device 24 and the first support member 30. Optionally, a second support member 26 can be disposed adjacent to the retaining device 24, between the retaining device 24 and the sealing member 28. The retaining device 24, the optional second support member 26, the sealing member 28 and the first support member 30 can be sized and dimensioned to fit into the packaged sensor 10. Thus, in the present embodiment, the foregoing components can comprise a substantially cylindrical shape, corresponding to the substantially cylindrical shape of the housing 12.

Each of the retaining device 24, the optional second support member 26, the sealing member 28 and the first support member 30 can comprise corresponding spaced apart channels 33a,b (shown in FIG. 3) adapted to support the electrical leads 32a,b therein in spaced apart relation and in electrical isolation.

In addition, the electrical leads 32a,b can be disposed co-axially in the corresponding spaced apart channels 33a,b such that they extend longitudinally from the retaining device 24 toward the seal 16. Thus, electrical leads 32a,b can extend longitudinally through one or more of the retaining device 24; optional second support member 26, sealing member 28, and first support member 30; and through channels 33a,b. If a connector is used, for example, then the leads 32a,b can terminate in the first support member 30. The leads 32a,b can comprise any electrically conductive material. For example, the leads 32a,b can comprise, metal strips and/or wires, and/or the like.

The optional second support member 26, the sealing member 28, and the first support member 30 can comprise any material capable of electrically isolating the components disposed in the housing 12, such as leads 32a,b, as well as can be capable of providing support for leads 32a,b. Possible materials for the first support member 30 and optional second support member 26 include, but are not limited to, ceramic, glass, and/or the like.

In addition, the material for the sealing member 28 can comprise any material capable of being compressed such that when compressed, can be capable of providing a seal or barrier to prevent fluid flow between the sensing end 10a and the terminal end 10b of the packaged sensor 10. When sealed, the fluid flow through the sealing member 28 can be less than or equal to 2 cubic centimeters per minute (2 cc/min) when the pressure on the sealing member 28 is greater than or equal to 50 pounds per square inch (psi). The material for the sealing member 28 can be disposed in the housing 12 as a compressible pre-form. Possible materials for the sealing member 28 include, but are not limited to, talc, glass, ceramic materials such as alumina, steatite, and the like, as well as combinations comprising at least one of the foregoing.

Referring now to FIG. 3, the retaining device 24 can comprise a first end 24a and a second end 24b. The retaining device 24 can comprise a body 34 defining a shoulder 40 disposed between the first end 24a and the second end 24b. Shoulder 40 can be tapered toward the first end 24a, and can be adapted to seat against the lower retention member 20 (e.g., crimped indentations) of the lower housing section 12a, when assembled. The retaining device 24 can comprise one or more channels adapted to receive and support one or more electrical leads therein. As shown, the retaining device 34 comprises two spaced apart channels 33a,b adapted to receive and support leads 32a,b, respectively, therein. This retaining device can have a length of less than or equal to about 20 mm, or more specifically, less than or equal to about 15 mm. The retaining device can comprise a material compatible with the exhaust environment and temperatures of up to 1,100° C., such as a ceramic material. Some exemplary ceramic materials that can be employed include steatite, aluminum oxide, and the like, as well as combinations comprising at least one of the foregoing ceramic materials.

A sensing element 36 can be disposed at the first end 24a of the retaining device 24 such that the terminal end (not illustrated) of the sensing element 36 can be in electrical communication with leads 32a,b. As shown, sensing element 36 is a planar sensing element, but it should be understood that other types of sensing elements could be used. The sensing element 36 can comprise any length suitable for the sensor in which it will be utilized. For example, the sensing element can comprise a length of less than or equal to about 40 millimeters (mm), more particularly less than or equal to about 30 mm, more particularly less than or equal to about 20 mm, and more particularly still less than or equal to about 15 mm. Electrical communication between the sensing element 36 and the leads 32a,b can be accomplished using various methods including, but not limited to, mechanical connections, welding, metallurgical bonding, wire harnesses, and/or the like. Metallurgical bonding allows the connection to be exposed to high temperatures, e.g., greater than or equal to about 900° C., while retaining electrical communication.

Optionally, the retaining device 24 can comprise a recessed region 35 disposed at the first end 24a of the retaining device 24 (as shown in phantom in the Figures), optionally comprising a sealant material 38. When the retaining device 24 comprises a recessed region 35, a portion of the sensing element 36 can be disposed in the recessed region 35, and the sealant material 38 can optionally be disposed around a portion of the sensing element 36 adjacent to the body 34 (e.g., around the terminal end). Thus, the recessed region 35 can contain the sealant material 38 and a portion of the sensing element 36. Thus, the retaining device 24 with the sealant material 38 can prevent or minimize fatigue at the connection region (the connection of the leads to contact pads on the terminal end of the sensing element).

The sealant material 38 can seal the connection region (i.e. the region at which the leads and sensing element are connected), which can be susceptible to contaminants, thereby inhibiting poisons from reaching the connection region. The sealant material 38 can also provide structural support for retaining the sensing element 36 in the retaining device 24 and/or in the recessed region 35, thereby minimizing or preventing movement of the sensing element 36 due to, for example, vibrations. The sealing material 38 can comprise a non-electrically conductive material that has a melting temperature of greater than the temperature in which the sensor will be employed (e.g., a melting temperature of greater than 1,100° C.). For example, the sensing material can comprise glass.

The body 34 of retaining device 24 can comprise any material capable of electrically isolating the leads 32a,b, and capable of providing support for the sensing element 36 and the leads 32a,b. Possible materials for the body portion 34 include the materials described above with regard to the optional second support member 26 and first support member 30, and the like.

Forming the packaged sensor 10 can comprise forming the retaining device 24 and disposing the retaining device 24 in the lower housing section 12a, such that the retaining device shoulder 40 is seated on the lower retention member 20, and such that the first end 24a of the sensing element 36 is disposed adjacent to the openings 14 of the lower housing 12a.

Optionally, after disposing the retention device 24 in the lower housing section 12a, the optional second support member 26 may be disposed therein. To do so, the leads 32a,b can be disposed in the second support member 26, and the second support member 26 can be disposed on the second end 24b of the retaining device 24, such that the leads 32a,b are disposed in electrical communication with the sensing element 36, and portions of the leads 32a,b extend from the opposite end of the second support member 26.

The leads can then be disposed in, for example, the spaced apart channels of the sealing pre-form (i.e. precursor to the sealing member 28, e.g. talc pre-form). The sealing pre-form then can be disposed in the lower housing section 12a such that it is supported on the terminal end 24b of the retaining device 24, and such that the leads 32a,b can be disposed in electrical communication with the sensing element 36. A portion of the leads 32a,b can extend from the opposite end of the sealing member 28, which can be attached to the terminals (not shown in the Figures).

Thereafter, the upper first support member 30 can be disposed such that it is supported on the sealing pre-form, and such that the leads 32a,b extend through the corresponding spaced apart channels 33a,b of the upper first support member 30. The upper housing section 12b can then be disposed over the upper first support member 30 until the upper retention member 22 engages the upper first support member 30, i.e., the downward movement of the upper housing section 12b is limited by the engagement of the upper first support member 30 and the upper retention member 22. As the upper housing section 12b is brought into engagement with the lower housing section 12a, the sealing member 28 can be formed by the longitudinal force exerted by the upper retention member 22 onto the upper first support member 30 and sealing member 28. The sealing member is longitudinally crushed between the retaining device 24 and/or the spacing member 26, and the upper first support member 30, thereby forming the sealing member 28.

After the upper and lower housing sections 12a,b are engaged, and the sealing pre-form 28 is crushed longitudinally, it also can be crimped to secure the upper and lower housing sections 12a,b together, and to further compact the sealing pre-form. After formation of the sealing member 28 is complete, it can prevent exhaust gases from traversing the packaged sensor from the openings 14. The sealing member also can prevent gases and containments from entering the housing from between the crimped region. When the talc is disposed above the crimped region, this pathway is prevented.

Optionally, one or more radial crimps may be used to secure the lower and upper housing sections—one to secure the upper and lower housing together, and another to optimize the talc compression for sealing.

For example, the sensor can comprise a housing having a length of greater than or equal to 70 mm, with a sensing element having a length of less than or equal to 40 mm disposed in a sensing end of the housing, wherein the sensing element can be metallurgically bonded to electrical leads at the element terminal end. The element terminal end can, optionally, be in physical communication with a sealant material of a retaining device, wherein the sealant material can comprise a glass material and can be disposed in a recessed portion of the retaining device. The electrical leads can extend through channels in the retaining device toward the housing terminal end. Various supports, e.g., support members, sealing members, and the like, can be disposed between the retaining device and the housing terminal end. This sensor can operate at temperatures of up to about 1,100° C.

The present sensor package: 1) can maintain the electrical leads in spaced relation and in electrical isolation; 2) can eliminate the use of wirebonding or brazing to make electrical attachments to ceramic elements; 3) can eliminate the use of mineral insulated cable and associated treatment steps for the cable; 4) can provide structural support for the sensing element; 5) can eliminate the use of metal mesh or cement to keep the element from vibrating (e.g., the various components (such as the retaining device, seal, can comprise ceramic material, talc, glass, and/or the like; 6) can eliminate the use of welding to seal the metal connections e.g. sealing the lower and upper housing together; 7) can provide a housing and flange that are a single, unitary member for mounting the sensor package in a vehicle exhaust system, thereby reducing the number of components (e.g., a separate flange that is welded to the housing), as well as a welding step; 8) can eliminate a possible leakage path (i.e., where the flange would otherwise be welded to the housing; 9) can provide a more secure attachment between the upper and lower housing sections; 10) can allow sensor use in temperatures up to about 1,000° C. (the present sensor can extend into the exhaust stream/exhaust treatment device (e.g., catalytic converter, oxidation catalyst, diesel particulate filter, trap, and/or the like) by a distance of greater than or equal to about 75 mm, or more; and 11) can have reduced sensor element lengths. The present sensor can employ a ceramic retaining device at the end of the sensor element and/or metallurgical bonds between the leads (that extend to the terminal end of the sensor housing) and the contacts on the sensor element terminal end, enabling it to withstand temperatures of about 900° C. to about 1,100° C.

Sensors employed throughout an exhaust system will generally have different lengths such that the sensing element can extend to an area that is to be sensed (i.e., a high temperature area of about 1,000° C. or greater), while the electrical connection to the sensing element is maintained outside of the high heat area. This required the production of a variety of sensing elements having different lengths. However, the sensing element disclosed herein can have a constant length (e.g., less than or equal to about 50 mm, or, more specifically, less than or equal to about 40 mm, or, even more specifically, less than or equal to about 30 mm), and be employed in any of the desired environments throughout the vehicle, and, in particular, the exhaust system. The sensor element length can be reduced by about 50 percent or more (e.g., from a length of greater than 60 mm (generally about 60 mm to about 65 mm or so for a sensor with a mechanical connection to the element) to a length of less than or equal to about 35 mm (e.g., about 20 mm to about 30 mm)). By reducing the length of the sensing element, the amount of metal (e.g., Pt, and the like) used for the leads can be reduced, thereby reducing the sensor cost.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A packaged sensor, comprising:
a housing defining a housing sensing end, a housing terminal end opposite the housing sensing end, and a chamber between the housing sensing end and the housing terminal end;
a retaining device disposed in the chamber, the retaining device comprising a first end and a second end, channels extending between the first end and the second end, and a sealant material disposed at the first end;
a sensing element comprising an element terminal end disposed in physical contact with the sealant material;
a sealing member disposed in the chamber between the retaining device and the housing terminal end;
a first support member disposed between the sealing member and the housing terminal end; and
a first electrical lead and a second electrical lead disposed in electrical communication with the element terminal end, extending from the element terminal end, through the channels in the retaining device, and extending from the second end of the retaining device.

2. The packaged sensor of claim 1, further comprising a flange extending radially from an exterior surface of the housing, wherein the flange and the housing are a single, unitary member.

3. The packaged sensor of claim 1, wherein the sensing element has a length of about 20 millimeters to about 30 millimeters.

4. The packaged sensor of claim 1, wherein the housing comprises a lower retention member and an upper retention member, and wherein the retaining device comprises a shoulder disposed between the first end and the second end, and adjacent to the lower retention member.

5. The packaged sensor of claim 1, wherein the electrical communication between the sensing element and the first and second electrical leads is provided by a metallurgical bond between the sensing element and the first and second electrical leads.

6. The packaged sensor of claim 5, wherein the sensing element has a length of less than or equal to about 35 millimeters.

7. The packaged sensor of claim 1, wherein the channels comprise a second channel spaced apart from a first channel, wherein the first channel is configured to support the first electrical lead, and the second channel is configured to support the second electrical lead in spaced apart relation from the first electrical lead.

8. The packaged sensor of claim 1, further comprising a recessed region disposed in the first end and extending toward the second end, and wherein the sensing element terminal end is disposed in the recessed region.

9. The packaged sensor of claim 1, wherein the retaining device comprises a ceramic material.

10. The packaged sensor of claim 1, wherein the housing further comprises a crimped region adjacent to the sealing member.

11. A method of making a packaged sensor, comprising:
disposing a retaining device in a chamber of a housing, wherein the housing comprises a housing sensing end, a housing terminal end opposite the housing sensing end and a chamber between the housing sensing end and the housing terminal end, and wherein the retaining device comprising a first end and a second end, channels extending between the first end and the second end, and a sealant material disposed at the first end;
disposing an element terminal end of a sensing element in physical contact with the retaining device;
disposing a sealing member in the chamber between the retaining device and the housing terminal end; and
disposing a first electrical lead and a second electrical lead in electrical communication with the element terminal end, wherein the first electrical lead and the second electrical lead extend from the element terminal end, through the retaining device, and from the second end of the retaining device.

12. The method of claim 11, wherein the housing is formed with a flange extending from an outer surface of the housing and wherein the housing with the flange is a single, unitary member.

13. The method of claim 11, wherein the sensing element has a length of less than or equal to about 40 millimeters.

14. The method of claim 11, wherein disposing the sealing member in the chamber further comprises disposing a sealing member precursor in the housing and exerting a longitudinal force on the sealing member precursor.

15. The method of claim 11, wherein the retaining device comprises a shoulder disposed between the first end and the second end, the housing comprises a lower retention member and an upper retention member, and further comprising disposing the shoulder of the retaining device adjacent to the lower retention member.

16. The method of claim 11, wherein disposing the first and second electrical leads in electrical communication with the sensing element further comprises forming a metallurgical bond between the electrical leads and the sensing element.

17. The method of claim 11, wherein the retaining device comprises a second channel spaced apart from the first channel, and further comprising disposing the first electrical lead in the first channel and the second electrical lead in the second channel.

18. The method of claim 11, further comprising disposing a recessed region and wherein the sensing element terminal end is disposed in the recessed region, and further comprising disposing a sealant material in the recessed region.

19. The method of claim 18, wherein the retaining device comprises a ceramic material, and the sealant material comprises glass having a melting temperature of greater than 1,100° C.

20. A packaged sensor, comprising:
a housing having a housing length of greater than 70 mm, wherein the housing defines a housing sensing end, a housing terminal end opposite the housing sensing end, and a chamber between the housing sensing end and the housing terminal end;
a ceramic retaining device disposed in the chamber, wherein the retaining device comprising a first end and a second end, a first channel extending between the first end and the second end, a second channel extending between the first end and the second end, and a sealant material disposed at the first end of the retaining device;
a sensing element having an element length of less than or equal to 40 mm wherein the sensing element comprises an element terminal end disposed in physical contact with the sealant material; and
a first electrical lead and a second electrical lead metallurgially bonded to the element terminal end, wherein the first electrical lead extends from the element terminal end through the first channel, and wherein the second electrical lead extends from the element terminal end through the second channel.
